(19) United States
(12) Patent Application Publication
(10) Pub. No.: US 2013/0138031 A1
(43) Pub. Date: May 30, 2013

(54) Stabiliser Assembly

(71) Applicant: Jeroen Henricus Maria Bergmann, Almere (NL)

(72) Inventor: Jeroen Henricus Maria Bergmann, Almere (NL)

(73) Assignee: BIOLOGICAL COPROCESSORS, INC., Somerville, MA (US)

(21) Appl. No.: 13/813,847

(22) PCT Filed: Aug. 2, 2011
(86) PCT No.: PCT/GB2011/051462

§ 371 Date: Feb. 1, 2013

(30) Foreign Application Priority Data
Aug. 2, 2010 (GB) 1012950.0

(51) Int. Cl. A61F 5/01

(52) U.S. Cl. CPC A61F 5/0195
USPC 602/28

(57) ABSTRACT

The present invention relates to an assembly to improve the stability of a user of the assembly, the assembly comprising: a brace for connecting to a lower leg portion, the brace comprising a stabiliser connecting portion; and a stabiliser for connecting to a shoe, the stabiliser comprising a brace connecting portion and an extension portion having a shoe connection portion configured in use to connect the extension portion to the shoe so that the extension portion extends laterally from the shoe to increase the effective surface area of the shoe sole at only a lateral side and to improve the stability of a user of the stabiliser, wherein the brace and the stabiliser are couplable together by the stabiliser connecting portion and the brace connecting portion so that, when said lower leg portion is connected to the brace and said shoe is connected to the stabiliser, an outward lean of said lower leg portion with respect to said shoe is limited to a predetermined angle, thereby providing a stabilising force to improve the stability of a user of the assembly, and wherein the stabiliser connecting portion and the brace connecting portion are uncoupled during normal locomotion of a wearer of the assembly, thereby providing unimpeded movement for normal locomotion.

### TECHNICAL FIELD OF THE INVENTION

The present invention relates to a stabiliser assembly comprising a brace and a stabiliser to help improve the stability of an individual, for example, an individual who may be prone to falling.

### BACKGROUND TO THE INVENTION

It is known to provide mobility aids to individuals who are prone to falling in order to improve their stability. Mobility aids are frequently provided to older adults who suffer from balance problems and are therefore more prone to falling.

The centre of mass (COM) of a body is the point at which the body's whole mass is considered to be concentrated and can be projected on the ground. The centre of mass of a human body is roughly located at the sacral promontory, anterior to S2. In order for a human body to remain stable its COM must fall within its base of support. The base of support of a human body is composed of the body's two feet and the area between them.

Traditional mobility aids, such as, for example, walking sticks, zimmer frames and rollators, operate on a principle of providing an individual with a hand-held device having points of contact with the ground which enlarge the individual's base of support. Using a mobility aid, an individual's base of support is defined by the mobility aid's points of contact with the ground, the individual's two feet and the area between all those points. By enlarging the individual's base of support in this way it becomes easier for the individual to keep their COM inside their base of support. This results in the individual being more stable.

One also needs to look at the ground reaction force in order to understand how the nervous system maintains the COM over the extended base of support. The ground reaction force is the sum of all the forces acting between the foot and the ground. It is a function not only of the position of the COM, but also the muscle actions at the joints of the body. The point of application of the ground reaction force on the floor is called the centre of pressure (COP).

If the body is perfectly still, the COP would lie exactly over the COM projection on the ground. However, in so called “static standing” there is always some postural sway, and by moving the COP and related ground reaction force the nervous system can control the sway. The important concept to grasp is that when this COP and the ground reaction force lie to one side of the COM, it causes the COM (and the body with it) to accelerate in the opposite direction. Thus, perturbations in the position of the COM can be corrected by carefully changing the COP.

Motion of the COP is a result of muscle actions in the body, and certain strategies are used preferentially by the sensorimotor system to maintain balance. In a left-right perturbation, the muscles around the ankle joint normally act first to control the unintended sideways motion. When this mechanism alone is insufficient to restore equilibrium, muscles around other joints will be activated to restore stability. If these mechanisms fail to restore balance a corrective step will be taken as soon as the COM falls outside the base of report.

In short, forces are needed to keep balance. However, the effectiveness of the generated force to maintain balance depends on the timing, magnitude and direction of the force. Furthermore, the distance between the generated force and the axis of the joint will also influence the end result. The combination of force and distance is defined as torque. A higher torque will counteract a perturbation more quickly when compared to lower torques.

### SUMMARY OF THE INVENTION

It is an advantage of the present invention to provide a stabiliser assembly comprising a stabiliser and a brace, for improving the stability of a user. The invention works as an additional torque generating system. It is a mechanical system and only provides an extra torque when needed. The system works by generating an external torque that is only generated when the COM is moving too far sideways and stability is compromised.

A first aspect of the present invention provides an assembly comprising:

a brace for connecting to a lower leg portion, the brace comprising a stabiliser connecting portion; and

a stabiliser for connecting to a shoe, the stabiliser comprising a brace connecting portion and an extension portion having a shoe connection portion configured in use to connect the extension portion to the shoe so that the extension portion extends laterally from the shoe to increase the effective surface area of the shoe sole at only a lateral side and to improve the stability of a user of the stabiliser,

wherein the brace and the stabiliser are couplable together by the stabiliser connecting portion and the brace connecting portion so that, when said lower leg portion is connected to the brace and said shoe is connected to the stabiliser, an outward lean of said lower leg portion with respect to said shoe is limited to a predetermined angle, thereby providing a stabilising force to improve the stability of a user of the assembly, and wherein the stabiliser connecting portion and the brace connecting portion are uncoupled during normal locomotion of a wearer of the assembly, thereby providing unimpeded movement for normal locomotion.

With regard to the stabiliser, the extension portion is preferably configured such that in use it extends laterally from only a portion of the length of the shoe sole at the lateral side.

Preferably, the extension portion is configured such that in use it extends laterally over from a middle portion and a rear portion of the length of the shoe sole at the lateral side.

Preferably, the extension portion is configured such that in use it does not extend laterally from a portion of the length of the shoe sole at the lateral side which is in-between the middle portion and the rear portion.

Preferably, the extension portion is configured such that in use it extends laterally from the full length of the shoe sole at the lateral side.

Preferably, the extension portion is configured such that in use it extends laterally from the shoe by about 2 cm to about 10 cm, and preferably by about 7 cm.

Preferably, the shoe connection portion comprises a shoe plate coupled to the extension portion, the shoe plate having an attachment element and being configured in use to connect to the shoe sole via the attachment element so that the extension portion extends laterally from the shoe to increase the effective surface area of the shoe sole at only the lateral side and to improve the stability of a user of the stabiliser. The attachment element may be any suitable means for attaching the shoe plate to the shoe sole. For example, the attachment portion may be one or more straps. The attachment portion holds the shoe plate in position against the shoe sole.

Preferably, the shoe plate is configured such that, when connected in use to the shoe sole, the shoe plate extends over only a portion of the shoe sole which is rearward of a metatarso-phalangeal joint region of the shoe, to facilitate normal locomotion by a wearer of the shoe.

Preferably, the shoe plate is configured such that, when connected in use to the shoe sole, the shoe plate extends over only a rear portion and a middle portion of the shoe sole.

Preferably, the shoe plate is configured such that, when connected in use to the shoe sole, the shoe plate extends over only a middle portion of the shoe sole.

Preferably, the shoe plate comprises a first rigid beam extending substantially normally from a lateral edge of the shoe plate and configured in use to abut a lateral side of the shoe, and a second rigid beam extending substantially normally from a medial edge of the shoe plate and configured in use to abut a medial side of the shoe, so that a portion of the shoe is received in-between the first and second rigid beams.

Preferably, at least one rigid beam is formed integrally with the shoe plate.

Preferably, the shoe plate comprises a recess arranged to receive a portion of the shoe.

Preferably, the attachment element comprises at least one strap for connecting the shoe plate to the shoe sole by clamping.

Preferably, the extension portion is formed integrally with the shoe plate.

Preferably, the stabiliser further comprises a second extension portion coupled to the shoe connecting portion such that in use the second extension portion extends medially from the shoe to increase the effective surface area of the shoe sole at only a medial side and to improve the stability of a user of the stabiliser.

Preferably, the second extension portion is configured such that in use it extends medially over only a middle portion of the length of the shoe sole at the medial side.

Preferably, the second extension portion is configured such that in use it extends medially from the shoe by about 0.5 cm to about 1.5 cm, and preferably by about 1.0 cm, to avoid impeding normal locomotion of a wearer of the stabiliser.

Preferably, the shoe plate further comprises a second extension portion coupled to a medial side of the shoe plate so that, when the shoe plate is connected in use to the shoe sole, the second extension portion extends medially from the shoe to increase the effective surface area of the shoe sole at only a medial side.

Preferably, the second extension portion is formed integrally with the shoe plate.

Preferably, wherein the extension portion extends laterally from the shoe so that the stabiliser increases the effective surface area of the shoe sole at only the lateral side.

Preferably, the extension portion and the second extension portion extend laterally and medially, respectively, from the shoe so that the stabiliser increases the effective surface area of the shoe sole at only the lateral side and the medial side.

Preferably, the stabiliser comprises an extension portion having a shoe connection portion configured in use to connect the extension portion to the shoe so that the extension portion extends laterally from the shoe to increase the effective surface area of the shoe sole at only a lateral side and to improve the stability of a user of the stabiliser; wherein the extension portion is configured such that in use it extends laterally from only a middle portion and a rear portion of the length of the shoe sole at the lateral side; wherein the shoe connection portion comprises a shoe plate coupled to the extension portion, the shoe plate having an attachment element and being configured in use to connect to the shoe sole via the attachment element so that the extension portion extends laterally from the shoe to increase the effective surface area of the shoe sole at only the lateral side and to improve the stability of a user of the stabiliser; wherein the shoe plate is configured such that, when connected in use to the shoe sole, the shoe plate extends over only a portion of the shoe sole which is rearward of a metatarso-phalangeal joint region of the shoe, to facilitate normal locomotion by a wearer of the shoe.

As indicated above, the assembly comprises: a brace for connecting to a lower leg portion, the brace comprising a stabiliser connecting portion; and a stabiliser for connecting to a shoe, the stabiliser comprising a brace connecting portion and an extension portion having a shoe connection portion configured in use to connect the extension portion to the shoe so that the extension portion extends laterally from the shoe to increase the effective surface area of the shoe sole at only a lateral side and to improve the stability of a user of the stabiliser, wherein the brace and the stabiliser are couplable together by the stabiliser connecting portion and the brace connecting portion so that, when said lower leg portion is connected to the brace and said shoe is connected to the stabiliser, an outward lean of said lower leg portion with respect to said shoe is limited to a predetermined angle, thereby providing a stabilising force to improve the stability of a user of the assembly, and wherein the stabiliser connecting portion and the brace connecting portion are uncoupled during normal locomotion of a wearer of the assembly, thereby providing unimpeded movement for normal locomotion.

The stabiliser can be couplable to the brace through the stabiliser connecting portion and the brace connecting portion in any suitable way so that, during normal locomotion (e.g. walking), the stabiliser connecting portion is in close proximity to the brace connecting portion but does not interact with it. In this way, during normal locomotion, the brace connecting portion does not exert any force on the stabiliser connecting portion and, therefore, does not impede the movement of the wearer of the assembly. This is an important advantage which previous mobility aids do not provide. It is not until the wearer leans outwards by more than the pre-determined angle, which might signify a loss of balance (i.e. a greater angle than would occur during normal locomotion), that the stabiliser connecting portion engages with the brace connecting portion, thus causing the brace to provide a force, or torque, on the stabiliser to help the wearer maintain his/her balance.

Normal locomotion is defined as the movement of the parts of the body of a person, in particular the legs, that normally occurs when a person moves from one place to another in a forward direction, i.e. the act of self-propulsion. This is the movement that occurs when a person is in complete control of said movement. It does not include the movement that occurs when a person is trying to regain control of his/her movement, e.g. when a person is trying to regain his/her balance. Typically, normal locomotion is walking but it may also include jogging, running, and other such forms of forward motion.

The coupling formed by the stabiliser connecting portion and the brace connecting portion may take a variety of forms. The stabiliser connecting portion and the brace connecting portion may be a hook and a loop, wherein the hook engages with the loop to provide the stabilising force and is disengaged from the loop during normal locomotion. Alternatively, the stabiliser connecting portion and the brace connecting portion may be a flexible wire or strap such that, during normal locomotion, the wire or strap is in a slack configuration so that the brace does not provide any force on the stabiliser. However, when an outward lean of the lower leg portion with respect to said shoe reaches the predetermined angle, the wire or strap is taut so that a stabilising force is provided to improve the stability of a user of the assembly. In another embodiment, a blocking cylinder can be used to generate the necessary force beyond a certain lean angle. The blocking cylinder consists of a piston that generates the resistance beyond a certain point of elongation. A hydraulic cylinder combined with a certain amount of give can be used to model a blocking cylinder. In these embodiments in which the brace connecting portion and the stabiliser connecting portion are under tension, i.e. the brace connecting portion exerts a ‘pulling’ force on the stabiliser connecting portion, the stabiliser connecting portion and the brace connecting portion will be positioned on the medial or inner side of the assembly.

Preferably, the stabiliser connecting portion and the brace connecting portion comprise a hook and a loop for receiving the hook.

Preferably, when said lower leg portion is connected to the brace and said shoe is connected to the stabiliser, an outward lean of less than the predetermined angle causes the hook to move freely with respect to the loop, thereby permitting normal locomotion of a wearer of the assembly.

Preferably, when said lower leg portion is connected to the brace and said shoe is connected to the stabiliser, an outward lean of the predetermined angle causes the hook to hook onto the loop thereby limiting the outward lean to the predetermined angle.

In one embodiment, the assembly comprises a resilient element which biases the stabiliser connecting portion and the brace connecting portion into the uncoupled position. For example, the resilient element may be a spring. However, the resilient element does not impede the normal locomotion of the user.

Preferably, the brace comprises a medial rigid panel for supporting a medial side of said lower leg portion, the medial rigid panel having at least one strap for connecting the medial rigid panel to the medial side of said lower leg portion, and wherein the hook extends from an end of the medial rigid panel which, in use, is adjacent to a foot-end portion of said lower leg portion.

Preferably, the brace further comprises a lateral rigid panel for supporting a lateral side of said lower leg portion, the lateral rigid panel being coupled to the at least one strap, and the at least one strap being further configured in use to connect the lateral rigid panel to the lateral side of said lower leg portion.

Preferably, the at least one strap comprises a first strap configured in use to connect the brace to a foot-end portion of said lower leg portion and a second strap configured in use to connect the brace to a knee-end portion of said lower leg portion.

Preferably, the or each strap comprises a connector to facilitate connection of the strap around said lower leg portion.

Preferably, when the stabiliser comprises a shoe plate, the loop comprises an n-shaped bar having both distal ends connected to a medial edge portion of the shoe plate, the loop being configured such that in use it extends from the shoe plate along the medial side of the shoe and towards the brace.

Preferably, the loop extends from a middle portion of the shoe plate.

Preferably, the loop is truncated and has a diameter in one direction of about 8 cm and a diameter in an orthogonal direction of about 4 cm.

Preferably, the assembly comprises: a brace for connecting to a lower leg portion, the brace comprising a medial rigid panel for supporting a medial side of said lower leg portion, the medial rigid panel having at least one strap for connecting the medial rigid panel to the medial side of said lower leg portion, the brace further comprising a stabiliser connecting portion; and, a stabiliser for connecting to a shoe, the stabiliser being as described above and further comprising a brace connecting portion, the brace and the stabiliser being couplable together by the stabiliser connecting portion and the brace connecting portion so that, when said lower leg portion is connected to the brace and said shoe is connected to the stabiliser, an outward lean of said lower leg portion with respect to said shoe is limited to a predetermined angle, thereby providing a stabilising force to improve the stability of a user of the assembly; wherein the stabiliser connecting portion comprises a hook and the brace connecting portion comprises a loop for receiving the hook, the hook extending from an end of the medial rigid panel of the brace which, in use, is adjacent to a foot-end portion of said lower leg portion, the loop comprising an n-shaped bar having both distal ends connected to a medial edge portion of the shoe plate, the loop being configured such that in use it extends from the shoe plate along the medial side of the shoe and towards the brace; wherein, when said lower leg portion is connected to the brace and said shoe is connected to the stabiliser, an outward lean of less than the predetermined angle causes the hook to move freely with respect to the loop, thereby permitting normal locomotion of a wearer of the assembly, and an outward lean of the predetermined angle causes the hook to hook onto the loop thereby limiting the outward lean to the predetermined angle.

A second aspect of the present invention provides an item of footwear comprising the assembly according to the first aspect.

### BRIEF DESCRIPTION OF THE DRAWINGS

Various example embodiments will now be described with reference to the following drawings, wherein like reference numerals relate to like components.

FIG. 1 is an asymmetric view of a first embodiment of a stabiliser;

FIGS. 2a and 2b are plan views of the stabiliser of FIG. 1. FIG. 2a shows the stabiliser with its straps in an open configuration whereas FIG. 2b shows the stabiliser with its straps in a closed configuration;

FIGS. 3a and 3b are plan views of second and third embodiments of stabilisers. FIG. 3a shows the second embodiment having a reduced size outward extension plate. FIG. 3b shows the third embodiment having a two-part outward extension plate and an inward extension plate;

FIGS. 4a, 4b and 4c are plan views of fourth, fifth and sixth embodiments of stabilizers, each embodiment having alternative shoe straps;

FIGS. 5a, 5b and 5c are rear views of seventh, eighth and ninth embodiment of stabilizers, each embodiment having alternative shoe connection arrangements;

FIGS. 6a and 6b illustrate a first embodiment of an assembly according to the present invention. FIG. 6a shows an asymmetric view of the assembly, whereas FIG. 6b shows a side view of a brace of the assembly;

FIGS. 7a and 7b are asymmetric views of the first embodiment of the assembly in use. FIG. 7a shows an outward side of the assembly whereas FIG. 7b shows an inward side of the assembly;

FIG. 8 illustrates the operation of the brace connecting portion and stabiliser connecting portion of the first embodiment of the assembly. FIG. 8a shows both connection portions during normal locomotion whereas FIG. 8b shows both connecting portions during an outward lean;

FIG. 9a illustrates the brace connection portion of the first embodiment of the assembly whereas FIG. 9b illustrates the brace connection portion of a second embodiment of the assembly;

FIG. 10 is a side view of a first embodiment of an item of footwear according to the present invention;

FIG. 11 is a graph showing the data obtained from an experiment to test the stabilising effect of the first embodiment of the assembly according to the present invention; and

FIG. 12 illustrates a second embodiment of an assembly according to the present invention.

### DETAILED DESCRIPTION OF EMBODIMENTS OF THE INVENTION

The present invention is intended to operate with the human body. In the following description the term ‘inward side’ will be used to describe the side of a foot, shoe or lower leg portion which lies on the inner side of the leg, i.e. the side of the leg which opposes the other leg when the person is standing upright. In other words the inward side is the medial side. When directly facing an upstanding person, the inward side of their left leg is the right side, and the inward side of their right leg is the left side. Further, the term ‘outward side’ will be used to describe the side of a foot, shoe or lower leg portion which lies on the outer side of the leg. In other words the outward side is the lateral side. When directly facing an upstanding person, the outward side of their left leg is the left side, and the outward side of their right leg is the right side. Furthermore, the term ‘forward’ will be used to describe the direction from the ankle towards the toe, and the term ‘rearward’ will be used to describe the direction from the ankle towards the heel.

In the following description, although a number of stabilisers are described in isolation of the brace, a skilled person will appreciate that each of the stabilisers described can also comprise a brace connecting portion and that the stabiliser can be combined with a brace having a stabiliser connecting portion to form the assembly of the invention.

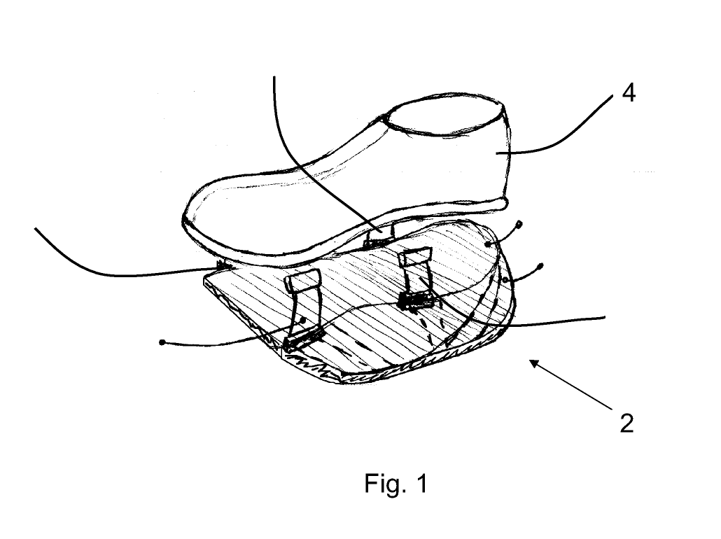

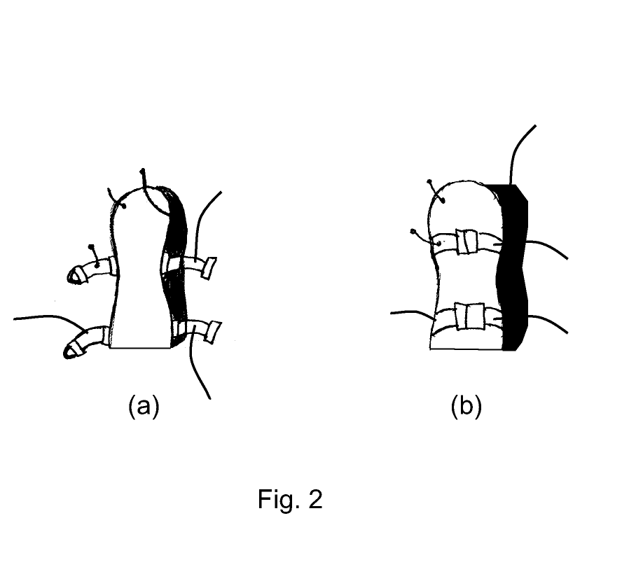

FIGS. 1, 2a and 2b show a first embodiment of a stabiliser 2. FIG. 1 also shows a shoe 4 with which the first embodiment is configured to operate. The stabiliser 2 comprises a shoe plate 6 which is configured to lie against an underside of the sole of shoe 4. The shape of shoe plate 6 substantially corresponds with the shape of a rearward portion of the sole of shoe 4. In particular, shoe plate 6 extends from the heel end of the shoe sole and terminates at a region of the shoe sole corresponding to where a metatarsal-phalangeal joint of a foot within the shoe 4 would lie.

The shoe plate 6 has an inward side which lies along the path of the inward side of the shoe 4. Coupled to an outward side of shoe plate 6 is an extension plate 8. The extension plate 8 comprises an extension portion of the present example embodiment. The extension plate 8 extends over the full length of the shoe plate 6, i.e. from the heel end of the shoe plate 6 to its most forward end. The extension plate 8 extends from the outward side of shoe plate 6 such that the surface area of shoe plate 6 at its outward side is increased. The extension plate 8 extends from the outward side of shoe plate 6 by about 0.5 cm to about 10 cm. Preferably, the extension plate 8 extends from the outward side of shoe plate 6 by about 2 cm to about 10 cm. More preferably, the extension plate 8 extends from the outward side of shoe plate 6 by about 5 cm to about 10 cm. More preferably still, the extension plate 8 extends from the outward side of shoe plate 6 by about 7 cm. In some embodiments, the extension plate 8 extends from the outward side of the shoe plate 6 by at least about 0.5 cm, preferably by at least about 2 cm, more preferably by at least about 5 cm, and even more preferably by at least about 7 cm. In some other embodiments, the extension plate 8 extends from the outward side of the shoe plate 6 by between about 25% to about 150% of the maximum width of the underside of the shoe sole. Preferably, the extension plate 8 extends from the outward side of the shoe plate 6 by between about 50% to about 150% of the maximum width of the underside of the shoe sole. Preferably still, the extension plate 8 extends from the outward side of the shoe plate 6 by between about 75% to about 125% of the maximum width of the underside of the shoe sole. In some embodiments, the extension plate 8 extends from the outward side of the shoe plate 6 by at least about 25% of the maximum width of the underside of the shoe sole, more preferably by at least about 50%, and even more preferably by at least about 75%. The term ‘about’ is taken to mean up to ±10%.

As seen more particularly on FIGS. 2a and 2b, the stabiliser 2 comprises four straps 10a to 10d. Each of the straps 10a to 10b has a proximal end attached to an edge of shoe plate 6. The straps 10a and 10b have their proximal ends attached to the outward side of shoe plate 6 whereas the straps 10c and 10d have their proximal ends attached to the inward side of shoe plate 6. A buckle is located at the distal end of each strap 10a to 10d. The straps 10a and 10d have corresponding buckles which are configured to attach together. The straps 10b and 10c also have corresponding buckles which are configured to attach together.

In operation, the shoe 4 is positioned on top of shoe plate 6 so that the shoe plate 6 overlays a rearward section of the shoe sole, and a toe portion of the shoe sole overhangs shoe plate 6. Straps 10a and 10d comprise front straps and may be connected together around the middle of shoe 4. The straps 10b and 10c comprise rear straps are attached together in a corresponding fashion around the shoe 4 rearward of the middle of shoe 4. According to this operation the straps 10a to 10d clamp shoe plate 6 against the underside of the sole of shoe 4. It is to be understood that the shoe connecting portion provides a rigid connection between the shoe and the stabiliser so that the stabiliser can provide a stabilising force to help stabilise the wearer. That said, the connecting portion does allow some flexibility in the rigid connection between the shoe and the stabiliser.

In this configuration, the forward end of shoe plate 6 terminates at the metatarsal-phalangeal joint region of shoe 4 and therefore, shoe plate 6 does not interfere with the normal bending of a foot inside shoe 4 which is necessary for the foot to perform normal locomotion. Further, extension plate 8 extends from the outward side of shoe plate 6 and thereby extends from the sole of shoe 4 to increase the surface area of the shoe sole at only the outward side. Accordingly, the base of support of a wearer of the stabiliser 2 is increased at an outward side. By increasing the base of support in this way the wearer has a greater area in which to keep their COM without loosing stability. Accordingly, the stabiliser 2 improves the stability of the wearer. Since the surface area at an outward side is increased by stabiliser 2, sideways balance in this direction is improved. The extension portion of the present embodiment is rigid so that it can provide a stabilising force to stabilise the wearer. However, the extension portion is flexible to some extent and may provide some resilience when loaded with the wearer's weight.

A feature of the stabiliser 2 which enables the wearer to perform normal locomotion is that the surface area of the shoe sole is only increased on the outward side. Stated differently, the surface area rearward of the shoe, forward of the shoe, or inward of the shoe are not affected by the stabiliser 2 to the extent that normal locomotion of the wearer is prevented. Therefore, the wearer does not have to change the way in which they walk or move.

Another feature of the stabiliser 2 which enables the wearer to perform normal locomotion is that the shoe plate only covers a portion of the shoe sole which is rearward of the metatarsal-phalangeal joint region. Therefore, the foot within the shoe is free to flex normally so that the wearer can walk and move normally.

The stabiliser 2 of FIGS. 1, 2a and 2b represents a left foot stabiliser since the extension plate 8 is positioned on the left outward side. It would be apparent to the skilled person how to modify the arrangement of stabiliser 2 to provide a right foot stabiliser. A left foot stabiliser when used in combination with a right foot stabiliser according to the present embodiment operates to increase the base of support of the wearer at the outward side of the wearer's left foot and the outward side of the wearer's right foot. The combination of both stabilisers improves the sideways balance of the wearer in both sideways directions since the base of support of the wearer is increased in both sideways directions.

The stabiliser according to the above-described embodiment provides a hands-free mobility aid. It is an advantage of the above-described embodiment that the wearer does not need to hold onto the stabiliser during use. This is particularly advantageous for wearers who have difficulty using their hands, such as rheumatoid arthritis patients. Another advantage is that the wearer's arms are free to manipulate objects, maintain balance and reduce injury severity should a fall occur. Conversely, users of traditional hand-held balance and mobility aids must hold onto the mobility aid in order to improve stability. A further advantage is that the stabiliser according to the above-described embodiment can be used with traditional mobility aids, since it is hands-free.

Since the stabiliser according to the above-described embodiment is attached to its wearer it does not require conscious thought for use. This is particularly advantageous for individuals suffering from dementia who, if using traditional hand-held mobility aids, may loose or forget the mobility aid.

The design of the stabiliser according to the above-described embodiment is unobtrusive since the only visible aspect is extension plate 8 which extends outward of the wearer's feet. An advantage of the stabiliser's unobtrusive design is to reduce the stigmatising effect felt by users of mobility aids, thereby increasing the user's self-confidence.

The stabiliser according to the above-described embodiment is not unwieldy to use. On the other hand, traditional hand-held mobility aids can be unwieldy making them unattractive for individuals, such as older adults, who do not have enough physical strength to manipulate the mobility device.

The stabiliser according to the above-described embodiment continues to improve balance and stability during transfers of weight necessary for performing normal locomotion. Conversely, traditional hand-held mobility aids require that an individual release the mobility aid in order to transfer their weight during normal locomotion and therefore, leave the individual unsupported when a fall is most likely to occur.

The stabiliser according to the above-described embodiment promotes normal locomotion and weight bearing since it does not require that the wearer modifies their walking motion. This advantage encourages the wearer to engage in physical activity and thereby promotes bone health. Such an advantage is particularly applicable to individuals with balance problems and medical conditions, such as cardiovascular disease. Conversely, traditional hand-held aids require that a user changes the way in which they walk in order to distribute some of their weight onto the hand-held mobility device. Consequently, at the start of a rehabilitation programme a patient must learn a new way of walking with the hand-held mobility aid, then once the patient is part-healed, the patient must change to moving without the mobility aid. This change in technique increases the time taken for the patient to return to normal thereby prolonging rehabilitation time. With the stabiliser according to the above-described embodiment, the patient can start moving normally from the start of the rehabilitation process.

Further, the sideways or lateral extension of extension plate 8 can also be specifically adapted to suit the needs of an individual. Therefore the stabiliser according to the above-described embodiment can be adjusted during rehabilitation to improve balance over a course of recovery. For example, at the start of a rehabilitation course the outward extension of outward extension plate 8 can be set relatively long then, as the course of rehabilitation progresses, the sideways extension can be gradually reduced until no extension is present. This can be achieved either by disconnecting the extension plate from the shoe plate and replacing it with a smaller version, or by shaving off portions of the extension plate to make it smaller. It is noted that in the above-described embodiment the shoe plate 6 is formed integrally with the extension plate 8, however, in some other embodiments both elements could be separate and attached by suitable means, such as, for example, mechanical fixings, adhesive, or welding.

In view of the above, use of the stabiliser according to the above-described embodiment is applicable in a number of different fields and for a number of individuals and situations. In particular, the stabiliser is suitable for older adults with balance problems, or individuals with neurological or degenerative pathologies, such as Parkinson's Disease and arthritis. The stabiliser is suitable for use by persons of all ages who have problems with their are stability or mobility, such as individuals with dyspraxia and neurological conditions. The stabiliser is suitable for use by individuals who have a fear of falling, reduced confidence with locomotion or who do not wish to use traditional mobility aids because of their social stigmas. The stabiliser can be used as a first line of defence against balance problems, i.e. it can be preventative rather than just curative. The stabiliser according to the above-described embodiment can be used with many different types of footwear. It can therefore be used to improve balance during participation in a variety of different sports and leisure pursuits, as well as being appropriate for use on a variety of different surfaces or terrains.

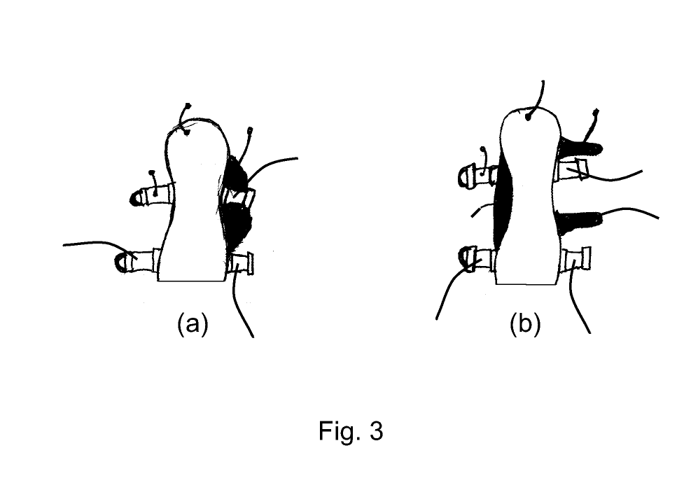

FIG. 3 shows second and third embodiments of the stabiliser. These alternative embodiments include alternative extension portions. FIG. 3a shows the stabiliser with an alternative outward extension plate 12 which only extends sideways from a middle portion of shoe plate 6. An advantage of this embodiment is that weight of the stabiliser 2 is reduced because less material is used in its construction. Furthermore, since extension plate 12 is smaller than extension plate 8 the design is less intrusive and therefore, further reduces the stigmatising effect of the stabiliser.

FIG. 3b shows a stabiliser with an alternative outward extension plate having two-portions 14a and 14b. The outward extension plate portion 14a extends from a rearward portion of side plate 6, whereas the outward extension plate portion 14b extends from a middle portion of side plate 6. Furthermore, the embodiment of FIG. 3b comprises an inward extension plate 16. The inward extension plate 16 comprises a second extension portion of the present example embodiment. The inward extension plate 16 extends from the inward side of shoe plate 6 at a middle portion thereof. The width (i.e. sideways extension) of the inward extension plate 16 is relatively small so that the inward extension plate 16 does not interfere with the normal locomotion of its wearer. Stated differently, the inward extension plate 16 is not large enough to hit or rub against the opposite foot or the inward extension plate 16 attached to the opposite foot. The width of inward extension portion 16 is between about 0.5 cm and about 1.5 cm. Preferably, the width of inward extension plate 16 is about 1.0 cm. In some embodiments the width of the inward extension portion 16 is less than about 1.5 cm, preferably less than about 1.25 cm, and more preferably less than about 1 cm. The term ‘about’ is taken to mean up to ±10%.

An advantage of the embodiment of FIG. 3b is that weight of the stabiliser 2 is reduced because the central portion of the outward extension plate is omitted. However, the effective surface area of the two-part outward extension plate is not reduced compared with extension plate 8 since the forward and rearward portions of extension plate 8 are the main aspects which improve stability. The effective surface area of the extension plate is the area between the furthest outlying points of contact with the ground of the extension plate. Considering the two-part extension plate, its effective surface area is the combination of the surface areas of both parts and the area between both parts. Another advantage of the embodiment of FIG. 3b is that it includes an inward extension plate to improve sideways balance in an inward direction, without affecting normal locomotion of the wearer.

It is noted that in some other embodiments only the two-part outward extension portion (14a and 14b) or the inward extension portion 16 is present. Furthermore, in some other embodiments either feature could be combined with features from other embodiments.

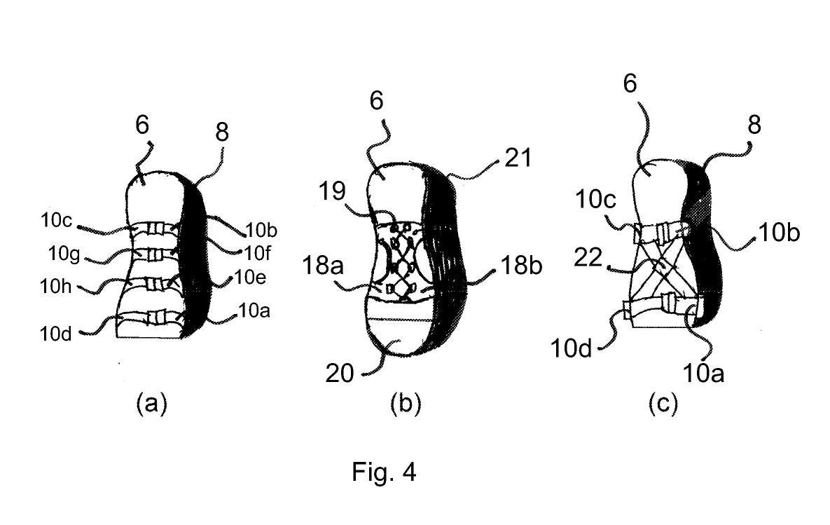

FIGS. 4a, 4b and 4c illustrate fourth, fifth and sixth embodiments of the stabiliser which each provide an alternative connecting portion by which the stabiliser connects with the shoe. FIG. 4a shows an embodiment in which four additional straps 10e to 10h are provided. Strap pairs 10e and 10h, and 10f and 10g operate in the same way as strap pairs 10a and 10d, and 10b and 10c, which are discussed above. An advantage of the embodiment of FIG. 4a is that the additional straps improve the connection between shoe plate 6 and shoe 4, which in turn improves the stabilising effect of the stabiliser.

FIG. 4b shows an embodiment in which straps 10a to 10d have been replaced by straps 18a and 18b, and lacing 19. The embodiment of 4b operates in the same way as previous embodiments, however, the shoe 4 is connected to shoe plate 6 by sliding shoe 4 under the laced together straps 18a and 18b, and then tightening the lacing 19 to clamp shoe 4 against shoe plate 6. The embodiment of 4b additionally provides toe plate 5 which extends from the forward end of shoe plate 6. Also, a modified outward extension plate 21 extends the full length of the shoe plate 6 and the toe plate 20. An advantage of the lacing arrangement of FIG. 4b is that its appearance is similar to that of shoe 4 and therefore, the design is less obtrusive. An advantage of providing toe plate 20 is to improve the wearer's balance while standing. Importantly, however, shoe plate 20 and toe plate 6 are joined using a flexible connection that extends laterally through the extension plate 21 to allow the wearer to flex their foot at the metatarsal-phalangeal joints. This permits normal locomotion

It is noted that in some other embodiments only one or two of the lacing arrangement (18a, 18b and 19), the toe plate 20 and the longer extension plate 21 is present. Furthermore, in some other embodiments each feature could be combined with features from other embodiments.

FIG. 4c shows an embodiment in which a cross shaped strap 22 is provided in-between strap pairs 10a and 10d, and 10b and 10c. The cross shaped strap 22 has four distal ends, each of which is attached to a different one of straps 10a to 10d. An advantage of the embodiment of FIG. 4c is that the connection between shoe 4 and shoe plate 6 is improved, which in turn improves the stabilising effect of the stabiliser.

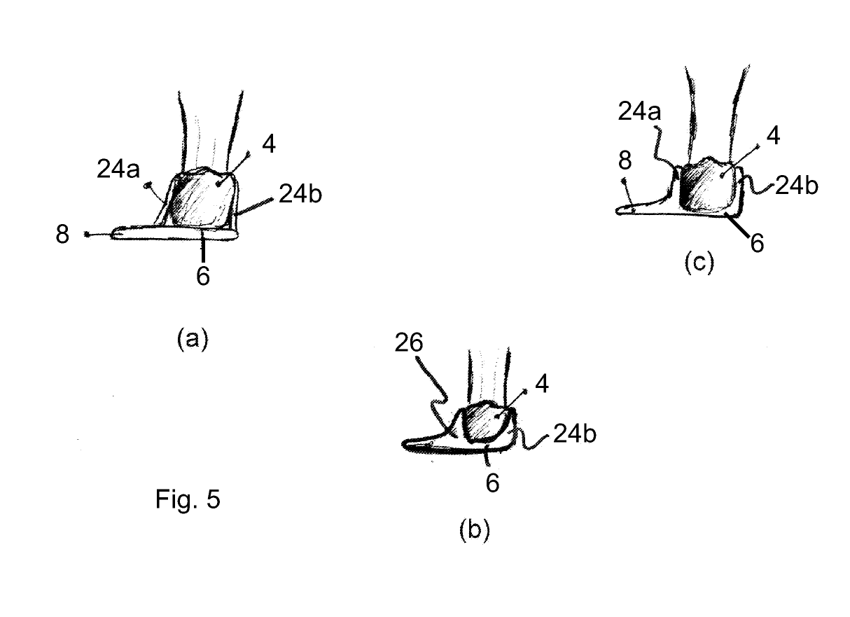

FIG. 5 shows seventh, eighth and ninth embodiments of the stabiliser, which provide alternative methods of connecting shoe plate 6 with shoe 4. The embodiment of FIG. 5a comprises two outer rigid beams 24a and 24b. The rigid beam 24a is connected to the outward edge of shoe plate 6 and the rigid beam 24b is connected to the inward edge of shoe plate 6. Both the rigid beams 24a and 24b extend upward from shoe plate 6. To account for the introduction of rigid beams 24a and 24b, the proximal ends of the straps 10a to 10d attach to the distal edge of the rigid beams 24a and 24b, rather than the outward and inward edges of shoe plate 6. In use, shoe 4 is received in-between rigid beams 24a and 24b so that straps 10a to 10d can be connected together as described above to secure the shoe 4 in-between rigid beams 24a and 24b, and to the shoe plate 6.

The embodiments of FIGS. 5b and 5c are variations of the embodiment of FIG. 5a. In the arrangements of FIGS. 5b and 5c the outward extension plate 8, the shoe plate 6, the outward rigid beam 24a and the inward rigid beam 24b are all formed integrally with each other. In FIG. 5b, the rigid beam 24a and the outward extension plate 8 are replaced by a wedge-shape cross-sectioned portion 26 which is formed integrally with shoe plate 6. The shoe plate 6 still comprises the inward rigid beam 24b. FIG. 5c shows a variation in which the rigid outer beam 24a of FIG. 5a is formed integrally with the outward extension plate 8 and the shoe plate 6. In FIGS. 5a and 5b, the integrated elements provide a recess designed to receive a portion of the shoe 4. Further, the shoe plate 6 of these arrangements is also formed with a recess in its upper surface for receiving the sole of the shoe 4. An advantage of the embodiments of FIG. 5 are that a more positive connection is made between the shoe 4 and the stabiliser 2. Accordingly, the stabiliser is more comfortable to wear and there is less movement of the shoe 4 within the stabiliser 2. Stated differently, the stabilising effect is improved. A further advantage of the wedge-shape cross-sectioned portion 26 is that it is stronger and therefore, it is more resistant to breaking during use.

In the embodiments of FIG. 5, the beams extend normally from the shoe plate 6, however, in some other embodiments they could extend at an angle of between 70° to 110°, or preferably between 80° to 100°, or more preferably between 85° to 95°.

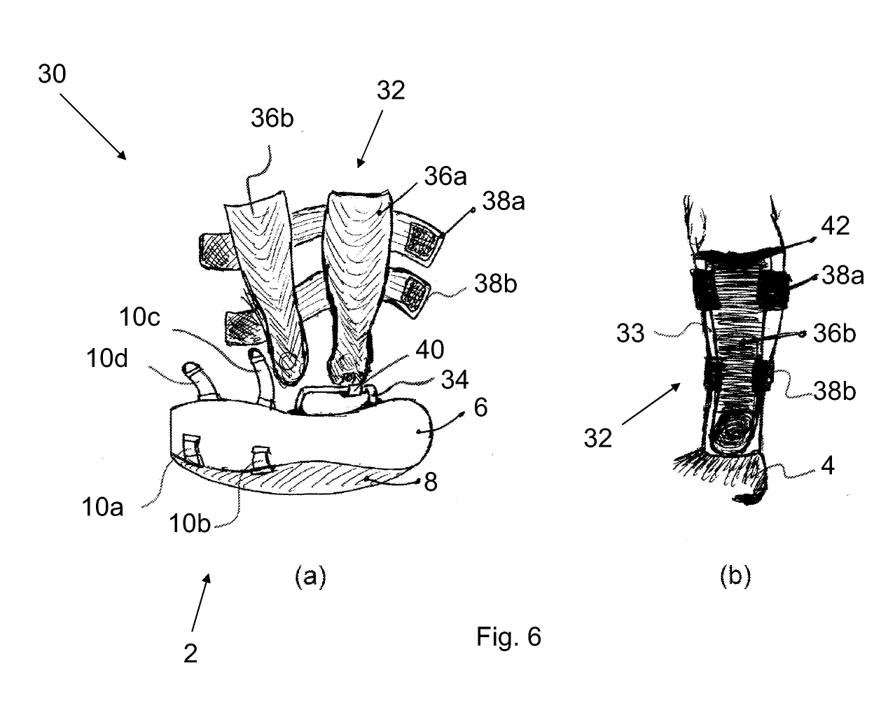

FIGS. 6a and 6b show an assembly 30 according to an embodiment of the invention. The assembly 30 comprises the stabiliser 2 and a lower leg brace 32. The stabiliser 2 is as described above, however, the stabiliser 2 further comprises a loop 34 extending from the inward edge of shoe plate 6. The loop 34 extends upward from the shoe plate 6 and extends along a central portion of the shoe plate 6. As shown more particularly in FIG. 6a, the loop 34 comprises an n-shaped bar having distal ends which provide connection points to the shoe plate 6. During operation, the loop will extend along the inward side of a shoe connected to the stabiliser 2. In the present example embodiment, the loop has a truncated shape and the loop's maximum diameter (measured along the length of the shoe plate 6) is about 6 cm to about 10 cm, and preferably about 8 cm. The loop's minimum diameter (measured upwards from the shoe plate 6) is about 2 cm to about 6 cm, and preferably about 4 cm. In some example embodiments, however, the size of the loop is dependent on the size of the stabiliser and, therefore, the loop size can be larger or smaller than the dimensions mentioned above.

As can be seen more particularly in FIG. 6b, the lower leg brace 32 comprises two rigid support panels 36a and 36b which are configured to support a lower leg portion 33 of a human wearer. In particular, inward panel 36a is configured to lie against and support an inward side of the lower leg portion 33, whereas outward panel 36b is configured to lie against and support an outward side of the lower leg portion 33. To ensure that rigid panels 36a and 36b effectively support the lower leg portion 33 both panels are profiled so that they follow the undulating surface of the lower leg portion 33. Two straps 38a and 38b are connected to panels 36a and 36b and are configured to hold panels 36a and 36b in position against the lower leg portion 33. In particular, upper strap 38a is attached to an upper portion of panel 36a and panel 36b whereas lower strap 38b is attached to a lower portion of panel 36a and panel 36b. Further, each strap 38a and 38b is provided with a connector, or connection means, so that it can be wrapped around the lower leg portion and connected onto itself to hold panels 36a and 36b in position. In the present example embodiment, the connection means are provided by hook and loop material. For each strap, a patch of hook material is positioned on one surface at one end, and a patch of loop material is positioned on the opposite surface at the opposite end.

Inward panel 36a additionally comprises a downward facing hook 40 at an end portion which is adjacent the lower end of the lower leg portion 33 when the brace 32 is fitted thereto. Furthermore, as seen more particularly on FIG. 6b, lower leg brace 32 may optionally comprise a secondary upper strap 42 to improve the connection between the lower leg brace 32 and the lower leg portion 33.

The following describes the operation of the assembly 30. The lower leg brace 32 is attached to the lower leg portion 33 of a wearer. In particular, inward panel 36a is positioned against the inward side of the lower leg portion 33 and outward panel 36b is positioned against the outward side of the lower leg portion 33. The straps 38a and 38b are then wrapped around the lower leg portion 33 and closed to locate and secure panels 36a and 36b in position against the lower leg portion 33.

Once the lower leg brace 32 is connected to the lower leg portion 33, the stabiliser 2 is fitted to the foot which is connected to the lower leg portion 33, as described above. In particular, the shoe 4 surrounding the foot is positioned against shoe plate 6 such that the heel of shoe 4 is on top of the rearward portion of shoe plate 6 and the toe portion of shoe 4 overhangs the forward end of shoe plate 6. The straps 10a to 10d are then attached in order to clamp shoe 4 to shoe plate 6 and thereby hold the sole of shoe 4 against shoe plate 6. In this configuration the extension plate 8 of stabiliser 2 extends outwardly from shoe plate 6 and the sole of shoe 4.

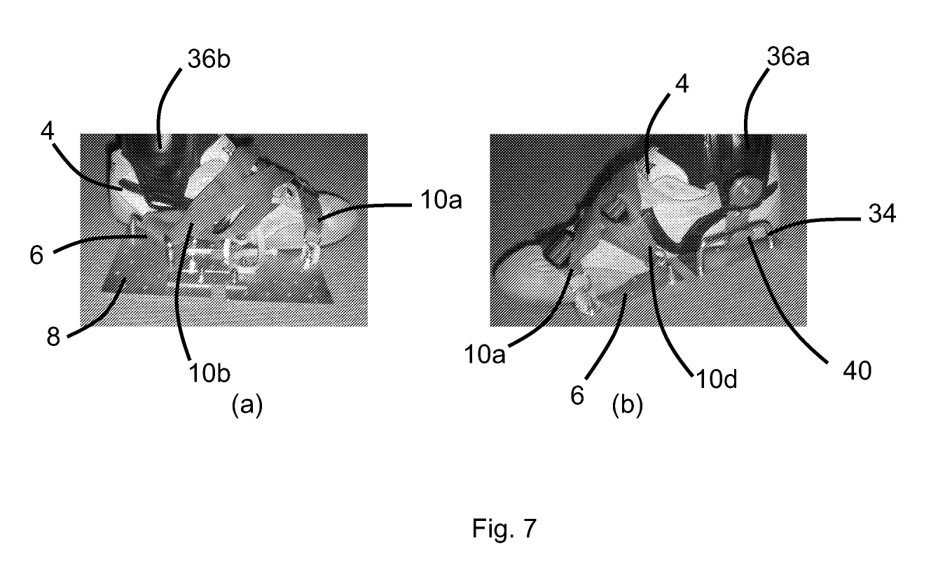

Finally, the wearer manipulates shoe 4 and lower leg portion 33 so that the hook 40 of lower leg brace 32 is received within the loop 34 of stabiliser 2 thereby connecting together the brace 32 and the stabiliser 2. This arrangement can be seen more particularly in FIG. 7, wherein FIG. 7a shows the outward side of the shoe 4 connected to stabiliser 2, and the lower part of the lower leg portion 33 connected to the brace 32. FIG. 7b shows the inward side of the shoe 4 connected to the stabiliser 2, and the lower part of the lower leg portion 33 connected to the brace 32.

It is to be understood that the above described assembly 30 relates to a right foot assembly. The skilled person would understand how to apply the above teaching to form a left foot assembly. Further, it is to be understood that in some example embodiments a wearer would only wear either the left-foot assembly or the right-foot assembly. However, in the present example embodiment the wearer is wearing a left-foot assembly on his left foot and a right-foot assembly on his right foot.

During operation, the extension plate 8 extends from the shoe plate 6 and the sole of shoe 4 to increase the surface area of the sole at the outward side. Since the wearer is wearing both a left-foot and a right-foot assembly, an extension plate 8 extends at the outward side of the wearer's left foot and right foot. By increasing the surface area of the left and right feet in this way the wearer's sideways stability is improved. In particular, the base of support of the wearer is increased at both outward sides.

The assembly 30 permits normal locomotion by the wearer. In particular, the forward end of shoe plate 6 terminates at a region of shoe 4 which corresponds to the position of the metatarsal-phalangeal joint of a foot within the shoe 4. Accordingly, the shoe plate 6 does not impede the normal foot flex necessary to perform normal locomotion.

A further feature of the assembly 30 which enables the wearer to perform normal locomotion is that the surface area of the shoe sole is only increased on the outward side. Stated differently, the surface area rearward of the shoe, forward of the shoe, or inward of the shoe are not affected by the stabiliser 2 to the extent that normal locomotion of the wearer is prevented. Therefore, the wearer does not have to change the way in which they walk or move.

In the following description the term ‘outward lean’ is used to describe a sideways lean of a wearer of the assembly 30. When facing a person who is standing-up straight, each of their legs extend upwards from its respective foot and roughly converge together at the top of both legs. In an outward lean to the right, the person's feet do not change position, however, both the person's legs extend upwards and to the right. In an outward lean to the left, the person's feet do not change position, however, both the person's legs extend upwards and to the left. A person can also perform an outward lean when standing on one leg and in such circumstances the direction of the lean is towards the outward side of that leg. When facing an upstanding person who is standing on their right leg only, an outward lean is towards the right. When facing an upstanding person who is standing on their left leg only, an outward lean is towards the left.

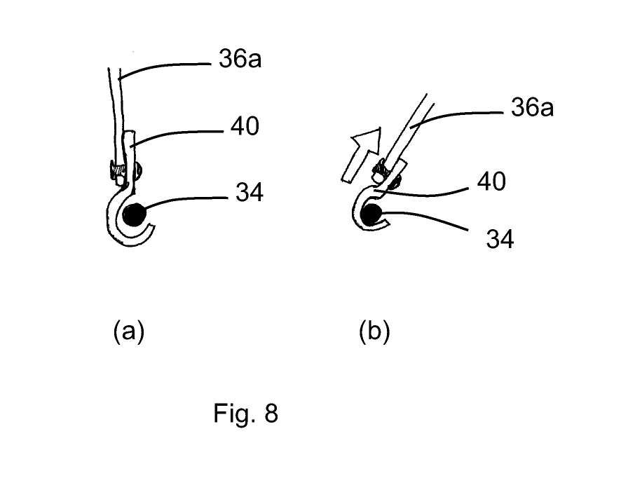

During normal locomotion the hook 40 is permitted to move freely in the loop 34. In this configuration the brace is not functionally connected to the stabiliser 2, i.e. neither element interferes with the movement of the other element. However, when the lower leg portion 33 leans outwardly with respect to the shoe 4, the hook 40 moves towards the top of the loop 34 and begins to hook onto the loop 34. As the outward lean increases the hook 40 firmly hooks the loop 34 thereby limiting any further outward lean. Stated differently, the hook 40 and loop 34 operate to limit the outward lean to a particular predetermined angle. In some embodiments the predetermined angle is between about 3° and about 30°, preferably between about 3° and about 20°, more preferably between about 3° and about 10°, and even more preferably about 5°. Since the brace 32 comprises rigid panels 36a and 36b which are firmly connected to the lower leg portion 33 by straps 38a and 38b, the brace 32 acts to provide an additional stabilising force which helps the wearer maintain balance. This operation can be seen more particularly in FIGS. 8a and 8b.

FIG. 8a shows a cross-section of a lower portion of panel 36a, the hook 40 and the loop 34 during normal locomotion. In this configuration, hook 40 moves freely within loop 34 and, the brace 32 and stabiliser 2 do not interfere with other's movement. FIG. 8b shows the same components as FIG. 8a, however, in FIG. 8b the wearer of the assembly 30 is performing an outward lean of the lower leg portion 33 with respect to shoe 4, which is of the predetermined angle. In this configuration, the hook 40 firmly hooks onto the loop 34 because the panel 36a is pulled away from the stabiliser 2. In particular, the panel 36a is attached to the inward side of the lower leg portion 33 and during an outward lean the gap between the bottom of panel 36a and the stabiliser 2 increases, whereas the gap between the bottom of panel 36b and the stabiliser 2 decreases. An effect of the above-described operation of hook 40 and loop 34 is to limit the size of the gap between the bottom of panel 36a and the stabiliser 2 thereby limiting the angle of outward lean to a predetermined amount. In this way the assembly 32 provides the wearer with an additional force with which to maintain balance. More specifically, the hook 40 is prevented from moving and, since the hook 40 is rigidly connected to rigid brace 32 which firmly connects to lower leg portion, the lower leg portion 33 is prevented from leaning further outward by the assembly 30.

It is to be understood that the brace according to the above-described embodiment is rigid enough to provide the additional force with which to maintain balance, however, the brace does allow for some flex in order to remain comfortable to wear against the lower leg portion during normal locomotion.

The above-described advantages of stabilisers according to various embodiments are also applicable to the assembly 30 when those stabilisers are part of the assembly 30. Further advantages of the assembly 30 are as follows.

It is an advantage of the assembly according to the above-described embodiment that the brace provides the wearer with additional support against falling sideways compared to when using the stabiliser alone. It is also an advantage that the brace does not affect the wearer's ability to perform normal locomotion because the brace only provides support when the wearer performs an outward lean of more than the predetermined angle. According to this operation, while support is not needed the brace allows full freedom of movement, and then when support is needed the brace limits movement in order to maintain sideways balance and improve sideways stability.

Since the brace is formed of rigid material and covers a majority of the lower leg portion, the brace can dissipate the holding force between the hook and the loop over most of the lower leg portion. The assembly therefore remains comfortable to wear.

Since the hook and loop connections are located on the inward side of the wearer's foot and lower leg portion, the appearance of these connecting portions is unobtrusive. Therefore, the wearer is less likely to feel self conscious while using the assembly, which in turn is likely to improve their self-confidence.

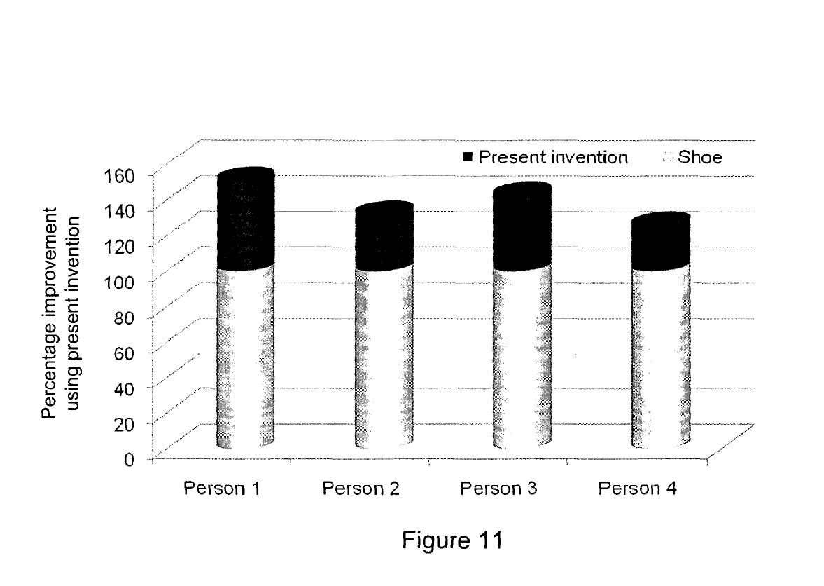

The following describes details of an experiment performed to show the stabilising effect achieved by the assembly 30. FIG. 11 graphically represents the data collected from the experiment. The experiment involved identifying a centre of pressure of a subject using a force plate. The force plate comprised a solid plate having four sensors attached to its bottom. The sensors were arranged to give an electrical output proportional to the force applied to the plate. In particular, the force plate consisted of four strain gauges which were connected to a USB-based analogue and digital I/O module (USB-1208FS, Measurement Computing Corporation, Norton, Mass., United States) combined with a software program written in Python WIN (Python WIN 2.4.3, Enthought Edition 1.0.0).

The activity of the subject's motor system can be assessed by measuring the location of the subject's centre of pressure using the force plate. An increased range of movement of the centre of pressure indicates a greater flexibility of the nervous system to respond to balance disruptions. A greater flexibility translates into improved stability of the subject. The centre of pressure can be defined as the point of application of a ground reaction force on the floor. The ground reaction force can be defined is the sum of all the forces acting between a subject's foot and the ground. The ground reaction force includes not only the position of the subject's COM, but also the muscle actions at the joints of the subjects body. By extending the base of support, the centre of pressure can move over a larger area providing more time to correct for unwanted motions of the COM.

Since assembly 30 aims to improve sideways balance, the force plate was used in the experiment to measure side to side movement of the subject's centre of pressure. During the experiment, subjects were asked to stand on the force plate on one leg and move as far as possible from one side to the other without losing balance.

Four people aged 22-57 years old were recruited to follow a randomised protocol in which they were wearing either their own flat sole shoes of preference or those shoes in combination with the assembly 30. Four measurements were taken for each condition and the best performance was chosen for further analysis. The range attained for wearing normal shoes was set to 100% in order to determine the percentage increase that was achieved by wearing the assembly 30.

As can be seen from FIG. 11, compared to normal shoes alone, the assembly 30 consistently improves balance in all participants, throughout all the measurements. The worst performance with the assembly 30 is significantly better than the best performance with only a shoe. Moreover, one volunteer's balance improved 52%. These results confirm that the assembly 30 improves sideways balance.

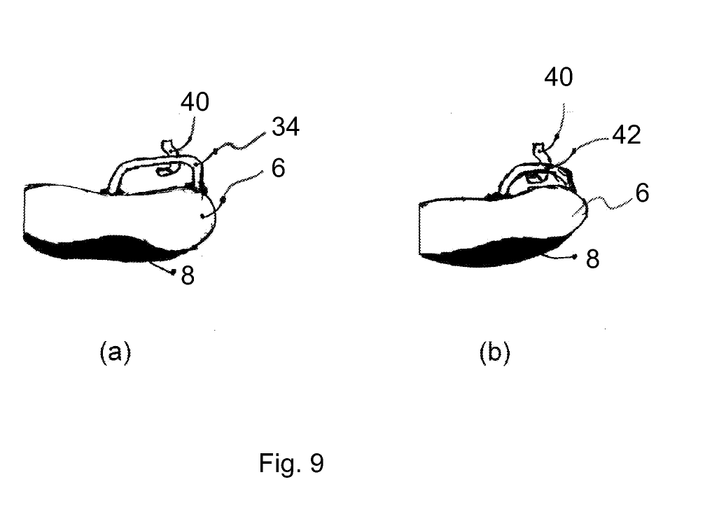

FIG. 9a shows the above-described stabiliser 2 of assembly 30. FIG. 9b shows a second embodiment of assembly 30, wherein an alternative loop arrangement is provided for stabiliser 2. In particular, loop 42 of FIG. 9b is smaller and more rounded than loop 34 of FIG. 9a. By adjusting the size and shape of the loop the predetermined angle at which outward lean is limited can be changed to limit the range of sideways motion of the wearer and control the wearer's stability. For example, increasing the size of the gap between the shoe plate 6 and the top of the loop will increase the predetermined angle. Conversely, decreasing the size of the gap between the shoe plate 6 and the top of the loop will decrease the pre-determined angle.

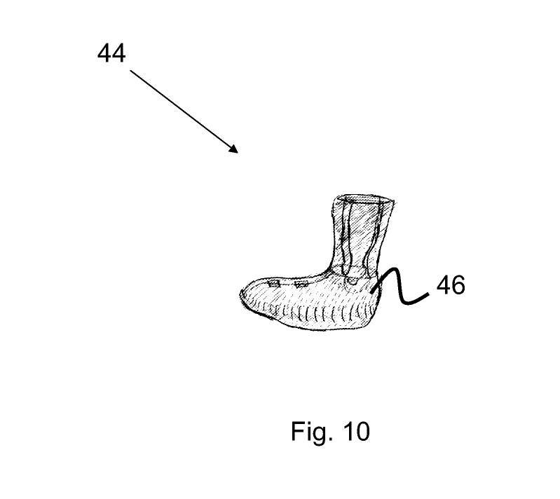

FIG. 10 shows a first embodiment of an item of footwear 44 according to the present invention. The item of footwear 44 comprises the assembly 30 and an outer fabric shell 46 which is arranged to cover the outside of the assembly 30 so that the whole unit 44 appears to be an item of footwear, e.g. a boot, rather than a shoe combined with a stabilising assembly. Accordingly, the footwear 44 provides an integrated solution by combining a shoe with the assembly 30. A feature of the item of footwear 44 is that the assembly 30 is concealed. An advantage of this feature is that the self confidence of the user is increased because the stigmatising effect relating to using mobility aids may be reduced.

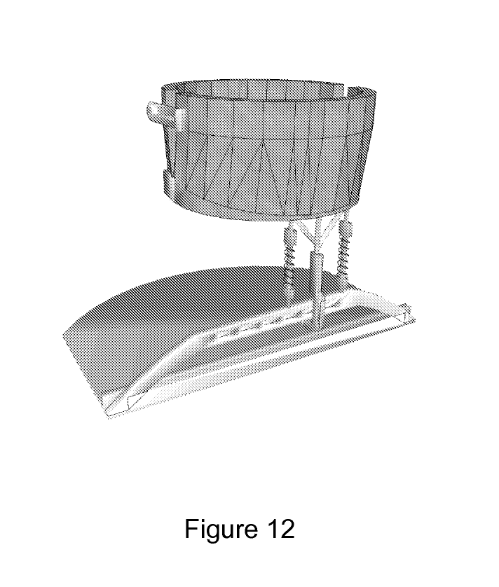

FIG. 12 illustrates a second embodiment of an assembly according to the present invention. This figure shows a particular connection mechanism between the brace and the stabiliser of the assembly. This connection mechanism is a spring-loaded hooking mechanism that connects to a small ankle brace. The connection mechanism generates an additional torque beyond a certain lean angle (angle between foot and shank). An elastic component (such as a spring loaded piston) can bring the system back to its original state. The spring also generates an incremental loading of the system. This connection mechanism does not affect normal human movement and only comes into effect when the range of movement falls beyond a critical value. The rigid brace provides a meaningful transfer of the forces.

Although the extension itself provides some stability, the dynamic connection to the brace (that cradles the shank), is the most important part of the invention, because this produces an additional external torque when needed. The connection system transfers a ground reaction force into an additional torque. The extension enhances the torque generated by the external actuator, which consist of a special connection mechanism between sole and brace. The mechanical connection allows free range of motion over the first two to three degrees and increases significantly over the subsequent degrees.

Various additions and modifications may be made to the above-described embodiments to form further alternative embodiments, any and all of which are intended to be encompassed by the scope of the appended claims.

In the above-described embodiments the loop is attached to the stabiliser and the hook is attached to the leg brace. However, in some alternative embodiments the hook may be attached to the stabiliser and the loop is attached to the lower leg brace. In some other embodiments connecting portions of other shapes could be used. For example, the hook could be replaced by a second loop (or ring) through-which the loop is received. Additionally or alternatively, the loop could be replaced by a curved or straight bar which is arranged to be received through the hook (or replacement loop or ring). Additionally or alternatively, in some other embodiments the brace and stabiliser are coupled together by a strap or wire, such as, for example, a fabric strap, a steel wire or a steel wire in a plastic sheath. According to these embodiments the brace connecting portion and the stabiliser connecting portion comprise the strap or wire and its connecting portions to the stabiliser and the brace. In operation, the strap or wire remains loose during normal locomotion but becomes taut during an outward lean of the predetermined angle. According to this operation, normal locomotion of a wearer of the assembly is not affected and an outward lean is limited to the predetermined angle. Embodiments are not limited to connecting portions of a particular shape. Rather the appended claims are intended to cover any structure which achieves the function of limiting an outward lean at a predetermined angle.

The precise shape of the extension portion may be different to the above-described embodiments, so long as the effective surface area at the outward side is increased. Changing the precise shape of the extension portion provides a means of changing the stabilising effect, its weight and its outward appearance. In the above-described embodiments an extension plate provides the extension portion. With the exception of the two-part extension plate, the effective surface area of the shoe fitted with the extension plate is the combination of the surface area of the shoe and the surface area of the extension plate. However, in some other embodiments, the extension portion is provided by something other than a flat plate. In some embodiments, the extension portion is provided by two substantially parallel bars extending normally from the outward side of the shoe, the distal ends of both bars being interconnected by another bar which is substantially parallel with the length of the shoe. According to this arrangement the effective surface area of the shoe fitted with this type of extension portion is the combination of the surface area of the shoe, the points of contact of the extension portion with the ground, and the area therebetween. It is noted that in this case the actual surface area of the extension portion will be less than its effective surface area. Furthermore, the effective surface area of this arrangement is the same regardless of whether or not the bars extending from the shoe contact the ground, as long as the interconnecting bar is arranged to contact the ground to improve stability. Therefore, with the exception of the two-part extension plate, in the above-described embodiments the surface area of the extension plate is the same as the effective surface area of the extension plate. References to ‘surface area’ in respect of these embodiments should therefore also be taken to include ‘effective surface area’. However, it is to be understood that in some other embodiments (including the two-part extension plate), the surface area of the extension portion is different from the effective surface area of the extension portion. In these cases, it is the effective surface area which is important for improving stability.

In the above-described embodiments the shoe plate is configured in use to cover the whole portion of the sole of the shoe which is rearward of the metatarso-phalangeal joint region of the shoe. In some other embodiments, the shoe plate may only cover part of that portion of the shoe sole. Alternatively, in some other embodiments the shoe plate may cover a larger portion of the shoe sole. In some other embodiments, the shoe plate may comprise one or more separate plates which are joined together by appropriate means, such as, for example, mechanical fixings, adhesive or welding. The separate plates may be arranged so that one or more gaps are present in-between the different plates. Such arrangements may be advantageous in improving the flexibility of the shoe plate so that it better mimics the underside of a shoe. Also, such arrangements may be lighter since less material may be used.

In the above-described embodiments the shoe plate is formed integrally with the extension plate. However, in some other embodiments the shoe plate can be a separate component to the extension plate and the two can be connected together by suitable means, such as, for example, mechanical fixings, adhesive or welding.

In the above-described embodiments the stabiliser is independent from the shoe. However, it is within the scope of some other embodiments that the stabilizer is formed integrally with the shoe. For example, in some of the other embodiments the shoe is provided with an integral outward extension plate extending from the outward side of the shoe sole at a portion of the shoe sole which is rearward of the metatarso-phalangeal joint region of the shoe.

In the above-described embodiments various materials may be used to manufacture the components described. For example, the shoe plate and extension plates may be made from a rigid lightweight material, such as, for example, aluminium or carbon reinforced plastic or some other fibre reinforced matrix material. Further, the plates may be rounded at the edges to improve safety. Further, the lower leg brace panels may be made of a rigid metal or plastic. Furthermore, the straps of the lower leg brace and the straps of the stabiliser 2 may be made of a fabric material which may be elasticised, but acts as a rigid component after a certain amount of stretch.

In the above-described embodiments the outward extension plate has been attached to the shoe by a shoe connecting portion comprising a shoe plate having straps. It is to be understood that in some other embodiments, the shoe connection portion could be different so long as the outward extension plate, when fitted in use, extends from the shoe to increase the surface area of the shoe sole at only an outward side. For example, the shoe connecting portion could comprise one or more pins which extend from the inward edge of the outward extension plate. The one or more pins could be arranged to fit against the underside of the shoe in a corresponding fashion to the shoe plate. The one or more pins could comprise straps which enable the one or more pins to be clamped to the shoe in a corresponding fashion to the above-described embodiments. Further, the one or more pins could be flattened so that a wearer of the shoe cannot feel them under their feet while walking. Alternatively, the pins could be arranged to penetrate the outward side of the shoe sole so that the outward extension plate extends directly from the outward side of the shoe sole. For example the one or more pins could be pointed at their distal end. Alternatively, the underside of the shoe sole could be provided with a lateral groove for receiving the one or more pins.

The above-described embodiments have been discussed with reference to a shoe. It is to be understood that the term ‘shoe’ encompasses all items of footwear, for example, boots, shoes, trainers, walking boots, mountaineering boots, tennis shoes and running shoes.

It is to be understood that one or more features of one of the above-described embodiments may be combined with one or more features from one or more of the other above-described embodiments to form alternative embodiments covered by the scope of the appended claims.

1. An assembly to improve the stability of a user of the assembly, the assembly comprising:
a brace for connecting to a lower leg portion, the brace comprising a stabiliser connecting portion; and
a stabiliser for connecting to a shoe, the stabiliser comprising a brace connecting portion and an extension portion having a shoe connection portion configured in use to connect the extension portion to the shoe so that the extension portion extends laterally from the shoe to increase the effective surface area of the shoe sole at only a lateral side and to improve the stability of a user of the stabiliser,
wherein the brace and the stabiliser are couplable together by the stabiliser connecting portion and the brace connecting portion so that, when said lower leg portion is connected to the brace and said shoe is connected to the stabiliser, an outward lean of said lower leg portion with respect to said shoe is limited to a predetermined angle, thereby providing a stabilising force to improve the stability of a user of the assembly, and wherein the stabiliser connecting portion and the brace connecting portion are uncoupled during normal locomotion of a wearer of the assembly, thereby providing unimpeded movement for normal locomotion.

2. The stabiliser of claim 1, wherein the stabiliser connecting portion and the brace connecting portion comprise: a hook and a loop for receiving the hook; a flexible wire; a strap; a blocking cylinder; or a piston.

3. The assembly of claim 1, wherein the extension portion is configured such that in use it extends laterally from only a portion of the length of the shoe sole at the lateral side.

4. The assembly of claim 2, wherein the extension portion is configured such that in use it extends laterally from only a middle portion and a rear portion of the length of the shoe sole at the lateral side.

5. The assembly of claim 3, wherein the extension portion is configured such that in use it does not extend laterally from a portion of the length of the shoe sole at the lateral side which is in-between the middle portion and the rear portion.

6. The assembly of claim 1, wherein the extension portion is configured such that in use it extends laterally from the full length of the shoe sole at the lateral side.

7. The assembly of any preceding claim, wherein the extension portion is configured such that in use it extends laterally from the shoe by about 2 cm to about 10 cm, and preferably by about 7 cm.

8. The assembly of any of claims 1 to 6, wherein the shoe connection portion comprises a shoe plate coupled to the extension portion, the shoe plate having an attachment element and being configured in use to connect to the shoe sole via the attachment element so that the extension portion extends laterally from the shoe to increase the effective surface area of the shoe sole at only the lateral side and to improve the stability of a user of the stabiliser.

9. The assembly of claim 7, wherein the shoe plate is configured such that, when connected in use to the shoe sole, the shoe plate extends over only a portion of the shoe sole which is rearward of a metatarso-phalangeal joint region of the shoe, to facilitate normal locomotion by a wearer of the shoe.

10. The assembly of claim 7, wherein the shoe plate is configured such that, when connected in use to the shoe sole, the shoe plate extends over only a rear portion and a middle portion of the shoe sole.

11. The assembly of claim 9, wherein the shoe plate is configured such that, when connected in use to the shoe sole, the shoe plate extends over only a middle portion of the shoe sole.

12. The assembly of any of claims 7 to 10, wherein the shoe plate comprises a first rigid beam extending substantially normally from a lateral edge of the shoe plate and configured in use to abut a lateral side of the shoe, and a second rigid beam extending substantially normally from a medial edge of the shoe plate and configured in use to abut a medial side of the shoe, so that a portion of the shoe is received in-between the first and second rigid beams.

13. The assembly of claim 10 or 11, wherein at least one rigid beam is formed integrally with the shoe plate.

14. The assembly of any of claims 7 to 12, wherein the shoe plate comprises a recess arranged to receive a portion of the shoe.

15. The assembly of any of claims 7 to 13, wherein the attachment element comprises at least one strap for connecting the shoe plate to the shoe sole by clamping.

16. The assembly of any of claims 7 to 14, wherein the extension portion is formed integrally with the shoe plate.

17. The assembly of any of claims 1 to 6, further comprising a second extension portion coupled to the shoe connecting portion such that in use the second extension portion extends medially from the shoe to increase the effective surface area of the shoe sole at only a medial side and to improve the stability of a user of the stabiliser.

18. The assembly of claim 16, wherein the second extension portion is configured such that in use it extends medially over only a middle portion of the length of the shoe sole at the medial side.

19. The assembly of claim 16 or 17, wherein the second extension portion is configured such that in use it extends medially from the shoe by about 0.5 cm to about 1.5 cm, and preferably by about 1.0 cm, to avoid impeding normal locomotion of a wearer of the stabiliser.

20. The assembly of any of claims 7 to 15, wherein the shoe plate further comprises a second extension portion coupled to a medial side of the shoe plate so that, when the shoe plate is connected in use to the shoe sole, the second extension portion extends medially from the shoe to increase the effective surface area of the shoe sole at only a medial side.

21. The assembly of claim 19, wherein the second extension portion is formed integrally with the shoe plate.

22. The assembly of any of claims 1 to 15, wherein the extension portion extends laterally from the shoe so that the stabilizer increases the effective surface area of the shoe sole at only the lateral side.

23. The assembly of any of claims 16 to 20, wherein the extension portion and the second extension portion extend laterally and medially, respectively, from the shoe so that the stabilizer increases the effective surface area of the shoe sole at only the lateral side and the medial side.

24. The assembly of claim 1, the stabiliser comprising an extension portion having a shoe connection portion configured in use to connect the extension portion to the shoe so that the extension portion extends laterally from the shoe to increase the effective surface area of the shoe sole at only a lateral side and to improve the stability of a user of the stabiliser;
wherein the extension portion is configured such that in use it extends laterally from only a middle portion and a rear portion of the length of the shoe sole at the lateral side;
wherein the shoe connection portion comprises a shoe plate coupled to the extension portion, the shoe plate having an attachment element and being configured in use to connect to the shoe sole via the attachment element so that the extension portion extends laterally from the shoe to increase the effective surface area of the shoe sole at only the lateral side and to improve the stability of a user of the stabiliser;
wherein the shoe plate is configured such that, when connected in use to the shoe sole, the shoe plate extends over only a portion of the shoe sole which is rearward of a metatarso-phalangeal joint region of the shoe, to facilitate normal locomotion by a wearer of the shoe.

25. The assembly of any preceding claim, wherein the stabiliser connecting portion and the brace connecting portion comprise a hook and a loop for receiving the hook.

26. The assembly of claim 25, wherein, when said lower leg portion is connected to the brace and said shoe is connected to the stabiliser, an outward lean of less than the predetermined angle causes the hook to move freely with respect to the loop, thereby permitting normal locomotion of a wearer of the assembly.

27. The assembly of claim 25 or 26, wherein, when said lower leg portion is connected to the brace and said shoe is connected to the stabiliser, an outward lean of the predetermined angle causes the hook to hook onto the loop thereby limiting the outward lean to the predetermined angle.

28. The assembly of any of claims 25 to 27, wherein the brace comprises a medial rigid panel for supporting a medial side of said lower leg portion, the medial rigid panel having at least one strap for connecting the medial rigid panel to the medial side of said lower leg portion, and wherein the hook extends from an end of the medial rigid panel which, in use, is adjacent to a foot-end portion of said lower leg portion.

29. The assembly of claim 28, wherein the brace further comprises a lateral rigid panel for supporting a lateral side of said lower leg portion, the lateral rigid panel being coupled to the at least one strap, and the at least one strap being further configured in use to connect the lateral rigid panel to the lateral side of said lower leg portion.

30. The assembly of claim 28 or 29, wherein the at least one strap comprises a first strap configured in use to connect the brace to a foot-end portion of said lower leg portion and a second strap configured in use to connect the brace to a knee-end portion of said lower leg portion.

31. The assembly of any of claims 28 to 30, wherein the or each strap comprises a connector to facilitate connection of the strap around said lower leg portion.

32. The assembly of any of claims 25 to 31, wherein the loop comprises an n-shaped bar having both distal ends connected to a medial edge portion of the shoe plate, the loop being configured such that in use it extends from the shoe plate along the medial side of the shoe and towards the brace.

33. The assembly of claim 32, wherein the loop extends from a middle portion of the shoe plate.

34. The assembly of claim 32 or 33, wherein the loop is truncated and has a diameter in one direction of about 8 cm and a diameter in an orthogonal direction of about 4 cm.

35. The assembly of claim 1 comprising:
a brace for connecting to a lower leg portion, the brace comprising a medial rigid panel for supporting a medial side of said lower leg portion, the medial rigid panel having at least one strap for connecting the medial rigid panel to the medial side of said lower leg portion, the brace further comprising a stabiliser connecting portion; and
a stabiliser for connecting to a shoe, the stabiliser comprising a brace connecting portion and an extension portion having a shoe connection portion configured in use to connect the extension portion to the shoe so that the extension portion extends laterally from the shoe to increase the effective surface area of the shoe sole at only a lateral side and to improve the stability of a user of the stabiliser
wherein the brace and the stabiliser are couplable together by the stabiliser connecting portion and the brace connecting portion so that, when said lower leg portion is connected to the brace and said shoe is connected to the stabiliser, an outward lean of said lower leg portion with respect to said shoe is limited to a predetermined angle, thereby providing a stabilising force to improve the stability of a user of the assembly; wherein the stabiliser connecting portion comprises a hook and the brace connecting portion comprises a loop for receiving the hook, the hook extending from an end of the medial rigid panel of the brace which, in use, is adjacent to a foot-end portion of said lower leg portion, the loop comprising an n-shaped bar having both distal ends connected to a medial edge portion of the shoe plate, the loop being configured such that in use it extends from the shoe plate along the medial side of the shoe and towards the brace; wherein, when said lower leg portion is connected to the brace and said shoe is connected to the stabiliser, an outward lean of less than the predetermined angle causes the hook to move freely with respect to the loop, thereby permitting normal locomotion of a wearer of the assembly, and an outward lean of the predetermined angle causes the hook to hook onto the loop thereby limiting the outward lean to the predetermined angle.

36. An item of footwear comprising the assembly as claimed in any of claims 1 to 35.
